(19) United States
(12) Patent Application Publication
(10) Pub. No.: US 2008/0262363 A1
(43) Pub. Date: Oct. 23, 2008

(54) MOBILE HEMODYNAMIC AND ELECTROPHYSIOLOGICAL INTERFACE TO PHYSIOLOGICAL MONITORS AND METHOD OF USE

(75) Inventors: Daniel Joseph Nowicki, Cedarburg, WI (US); Scott Raymond Kosloske, Slinger, WI (US); Sachin Vadodaria, Cedar Park, TX (US)

Correspondence Address: MCANDREWS HELD & MALLOY, LTD, 500 WEST MADISON STREET, SUITE 3400, CHICAGO, IL, 60661, US

(73) Assignee: General Electric Company, Schenectady, NY (US)

(21) Appl. No.: 11/737,886

(22) Filed: Apr. 20, 2007

(51) Int. Cl. A61B 5/02; A61B 5/00

(52) U.S. Cl. 600/509; 600/300

(57) ABSTRACT

Certain embodiments provide systems and methods for patient monitoring using a mobile workstation and one or more patient monitors. Certain embodiments include selecting a patient monitor via a mobile physiological monitoring workstation; acquiring physiological data from the patient monitor; and displaying the physiological data at the mobile physiological monitoring workstation. The data is transmitted over a network from the patient monitor to the mobile physiological monitoring workstation without a dedicated connection between the mobile physiological monitoring workstation and the patient monitor. Certain embodiments include at least one patient monitor capable of obtaining and transmitting physiological data for a patient; a mobile physiological monitoring workstation receiving physiological data; and a network enabling exchange of physiological data between the at least one patient monitor and the workstation. The workstation may select one or more of the at least one patient monitor from which to receive physiological data for the patient.

### BACKGROUND OF THE INVENTION

The present invention generally relates to interfacing between an electrophysiology/hemodynamic (EP/Hemo) workstation and a physiologic monitor. More specifically, certain embodiments of the present invention related to systems and methods for interfacing between multiple monitoring devices and an EP/Hemo workstation.

During clinical invasive procedures, such as interventional cardiology or radiology procedures, there is a need to continuously monitor physiological parameters of a patient. Monitoring a patient is done using physiological monitoring and recording systems, such as the GE Mac-Lab for hemodynamic procedures and the GE CardioLab for electrophysiology procedures.

Hemodynamic monitoring can aid in detection, identification, and treatment of life-threatening conditions such as heart failure and cardiac tampanade. Using invasive hemodynamic monitoring, for example, a practitioner can help evaluate a patient's response to treatment, such as drugs and mechanical support. A practitioner can evaluate the effectiveness of cardiovascular function such as cardiac output and cardiac index.

Electrophysiological data includes an analysis of the electrical conduction system of a patient's heart, which generates a heart beat. Catheters may be inserted in a vein and are then passed into the heart under fluoroscopic guidance, for example. The catheters measure the electrical signals generated by the heart to obtain a more detailed analysis of the electrical signals than a simple surface electrocardiogram (ECG).

Invasive and/or noninvasive techniques can be used to determine hemodynamic and/or electrophysiological data for a patient. For example, a patient's blood pressure may be measured using a cuff, and/or pressure within a heart may be measured invasively using a catheter. Blood and/or heart pressure measurement may include a systolic pressure and a diastolic pressure. Using the two measurements, a mean pressure can be calculated. Parameters such as chest cardiac output (CO), cardiac index (CI), pulmonary artery wedge pressures (PAWP), and cardiac index (CI) may be measured using a catheter.

In order to track issues and to determine the effectiveness of a catheterization procedure, a patient's vital signs are logged before and after the procedure into an EP/Hemo workstation. Typically, logging of vital signs is done in a different room from the catheterization procedure with a patient monitoring device. Vital signs data is currently either manually entered or collected with a dedicated one-to-one serial communication link. This configuration requires multiple catheterization monitoring workstations, one per pre-/post-operating room, thus increasing equipment cost and space requirements.

To minimize space and equipment costs, among other things, it would be highly desirable to allow a user to link to multiple monitoring devices from one catheterization workstation. There is a need for systems and methods for interfacing between an EP/Hemo system and one or more monitoring devices.

### BRIEF SUMMARY OF THE INVENTION

Certain embodiments provide systems and methods for patient monitoring using a mobile workstation and one or more patient monitors.

Certain embodiments provide a method monitoring patient physiological data. The method includes selecting a patient monitor via a mobile physiological monitoring workstation. The method also includes acquiring physiological data from the patient monitor. The data is transmitted over a network from the patient monitor to the mobile physiological monitoring workstation without a dedicated connection between the mobile physiological monitoring workstation and the patient monitor. The method further includes displaying the physiological data at the mobile physiological monitoring workstation.

Certain embodiments provide a patient monitoring system. The system includes a plurality of patient monitors. The plurality of patient monitors communicate via at least one of wired and wireless communication links. Each of the plurality of patient monitors is capable of obtaining physiological data for a patient. The system also includes a wireless mobile physiological monitoring workstation receiving physiological data. The system further includes a network enabling exchange of physiological data from the plurality of patient monitors and the workstation. The workstation may select one or more of the plurality of patient monitors from which to receive physiological data for the patient.

Certain embodiments provide a patient monitoring system. The system includes at least one patient monitor capable of obtaining and transmitting physiological data for a patient. The system also includes a mobile physiological monitoring workstation receiving physiological data. The system further includes a network enabling exchange of physiological data between the at least one patient monitor and the workstation. The workstation may select one or more of the at least one patient monitor from which to receive physiological data for the patient.

### BRIEF DESCRIPTION OF SEVERAL VIEWS OF THE DRAWINGS

FIG. 1 illustrates a patient monitoring system in accordance with an embodiment of the present invention.

FIG. 2 illustrates a flow diagram for a method for communication between a mobile workstation and a plurality of patient monitors in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Certain embodiments of the present invention provide an electrophysiology and hemodynamic (EP/Hemo) and/or other physiological recording or monitoring system with an ability to receive and transmit data, including physiological signal data, with one or more patient monitors. Certain embodiments provide methods for exchanging physiological signal data between an EP/Hemo system and a patient monitor or other similar system. The signal data may be electrocardiogram (ECG) and/or other waveform data, for example.

An EP/Hemo system obtains EP and/or hemo data, for example, for one or more patients. In order to share information, the EP/Hemo system may provide one or more interfaces to different hemodynamic systems, electrophysiological systems, catheterization lab systems, and database systems, for example. The information can be collected before, during and/or after a catheterization procedure and may be shared with laboratory and hospital repository systems (e.g., orders and results) for a patient record. Interface(s) may be based on industry-standard protocols (e.g., HL7, SQL, ASCII) and/or specific interface(s) for systems that do not support standard protocols, for example. The interface(s) allow exchange and sharing of data (e.g., demographics, history, log, results etc.) between different systems and vendors, for example.

The EP/Hemo system can combine hemodynamic and electrophysiological monitoring into a single system configuration to allow dual use of a catheterization or other lab. EP and hemo data can be stored in a single database to help streamline documentation and access to patient information. The EP/Hemo system provides laboratory performance and resources for patient care. In certain embodiments, the EP/Hemo system may be used in one or more locations, as well as in transit, for example. In certain embodiments, the EP/Hemo system may be accessed remotely.

In certain embodiments, the EP/Hemo system includes a graphical user interface to facilitate user-defined procedural lists, macros and configurable electronic documentation. The EP/Hemo system may include a multi-parameter module, such as a GE TRAM® module, that acquires and processes patient physiological parameters, such as ECG, invasive blood pressure, non-invasive blood pressure, pulse oximetry, cardiac output, temperature, respiration, etc. Patient data may be measured in real-time and/or substantially real-time, for example. The EP/Hemo system may also be configured for administrative reporting and facilitation of clinical workflow. The EP/Hemo system may further provide on-line help resources and an ability to save data to a network and/or attached storage, for example.

The EP/Hemo system may include a variety of inputs/outputs, such as one or more ECG leads, one or more stimulation inputs, one or more invasive pressure signals, one or more recording channels, one or more intracardiac channels, one or more catheter inputs, etc. The EP/Hemo system provides diagnostic tools, as well as intracardiac and ECG recording capability, for example. In certain embodiments, the system provides bi-polar channel scalability, automated clinical features and activation mapping to aid in diagnosis. The system may provide a 3D mapping interface as well as connectivity to external system(s), for example. In certain embodiments, the EP/Hemo system may interface uni- or bi-directionally with another system, such as a navigation and/or ablation system to share information, such as mapping events, clinical data and/or EP report data. The EP/Hemo system may be configured to operate in a plurality of languages.

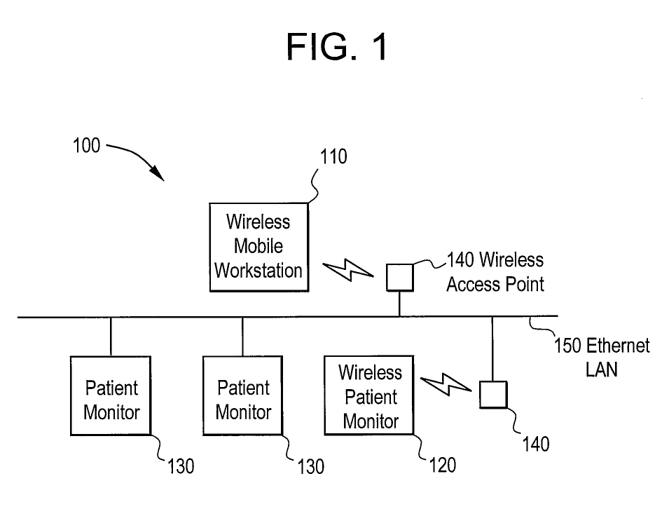

FIG. 1 illustrates a patient monitoring system 100 in accordance with an embodiment of the present invention. The system 100 includes a wireless mobile workstation 110 and one or more patient monitors, such as a wireless patient monitor 120 and other wired/wireless patient monitor(s) 130. The workstation 110 and monitor(s) 120, 130 communicate via a network 150. The wireless workstation 110 and wireless monitor 120 use one or more wireless access points 140 to communicate via the network 150.

In certain embodiments, the workstation 110 is an EP/Hemo system workstation, for example. In certain embodiments, the network 150 is a local area network, such as an Ethernet network. The network 150 may be a wired and/or wireless network, for example. Connections between the workstation 110 and the monitor(s) 120, 130 may be wired and/or wireless via the network 150, for example. In certain embodiments, one or more wireless access points 140 facilitate communication between workstation 110 and monitor(s) 120, 130 via the network 150. For example, the wireless access point 140 may serve to link the wireless workstation 110 and wireless monitor 120 to an Ethernet network.

In certain embodiments, the wireless access point 140 is a device that connects wireless communication devices together to form a wireless network. The wireless access point 140 may connect to a wired network 150 and can relay data between wireless devices 110, 120 and wired devices 130, for example. In certain embodiments, several wireless access points 140 can link together to form a larger network that allows “roaming”. In certain, wireless access points 140 have IP addresses for configuration.

The system 100 utilizes an interface between the workstation 110, such as an EP/Hemo workstation, and patient physiologic monitor(s) 120 and/or 130 to collect physiological and/or other patient data, such as vital sign information, for a patient before and after a procedure, such as a catheterization case. Waveform and/or other patient data may also be collected from the patient monitor 120, 130, for example.

In certain embodiments, the patient or medical monitor 120, 130 is a manual or automated medical device that senses a patient's vital signs and/or other data and displays the results. In certain embodiments, monitors 120, 130 allow for continuous supervision of a patient without continuous attendance, thus improving patient care. In certain embodiments, monitors resemble oscilloscopes and/or computer monitors to obtain and display data. Additionally, some monitors (e.g. ECG and EEG) are in contact with patients and obtain data from the patient. In certain embodiments, monitors 120, 130 may be specialized to track and/or measure particular data, such as a patient's blood pressure, pulse oximetry, etc. In certain embodiments, monitors 120, 130 may be multi-parameter monitors that can track/measure a plurality of data at once and/or be programmed to track/measure a series of different data at different times, for example.

In operation, a clinical user may select a patient-monitor data source 120, 130 at the workstation 110 by selecting from one or more criteria such as physical location, patient name, patient identifier, etc. Data may be retrieved from the wireless patient monitor 120, other patient monitor 130 and/or other data source via the network 150 for viewing and/or processing at the workstation 110.

Thus, certain embodiments provide a network connection and obviate a need for a dedicated, serial link. Using the network connection, the mobile workstation 110 may link to any patient monitor 120, 130 on the network 150 rather than being tied to one patient monitor. Using a wireless network connection 140, the mobile workstation 110 may be moved to a location convenient for a particular workflow. Certain embodiments help reduce cost and space requirements for a clinical environment by providing a mobile workstation and flexible, mobile monitor connectivity. A network may be used to share data with central monitoring stations and clinical information systems as well as EP/Hemo workstations, for example.

For example, as described above, data, such as physiological waveform data, is acquired from a patient or external system, such as an EP/Hemo system. The data is transmitted from the EP/Hemo system via the signal output port, for example. The waveform data is transmitted to a patient monitor 120, 130 ultrasound system 230 via the network 150. Similarly, data may be communicated from a patient monitor 120, 130 to a system, such as an EP/Hemo system via the network 150.

In certain embodiments, an external systems, such as an EP/Hemo system may include a processor for processing and/or storage waveform and/or other data, for example. The system may also include an interface for facilitating communication and/or data transfer between the system and the patient monitor 120, 130 via the network 150. The external system may also include an analog signal output and one or more single and/or multi-parameter measurement monitors for obtaining, processing and/or relaying physiological and/or other data for a patient, for example. The external system may be a fixed and/or mobile workstation having one or more wired and/or wireless communication ports/connections, for example.

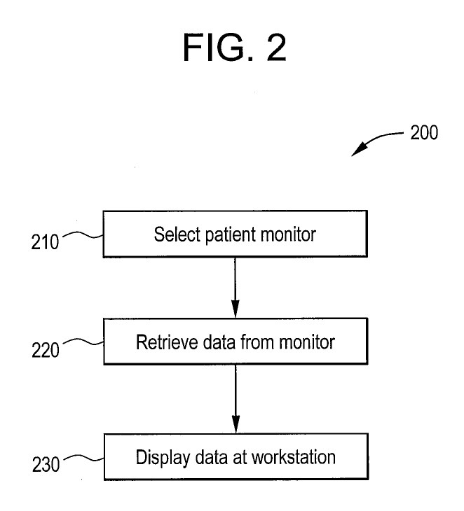

FIG. 2 illustrates a flow diagram for a method 200 for communication between a mobile workstation and a plurality of patient monitors in accordance with an embodiment of the present invention. At step 210, a patient monitor data source is selected at the workstation. The patient monitor source may be selected manually by a user and/or automatically by a software program, for example. For example, a user at a workstation 110 in an operating room may access a monitor 120 to request patient blood pressure data prior to a procedure. In certain embodiments, one or more monitors may be selected based on one or more criteria such as physical location, patient name, patient identifier, etc.

At step 220, data is retrieved from the selected patient monitor via a network. Data may be transferred via one or more wireless and/or wire-based connections, for example. Data may be retrieved from the wireless patient monitor 120, other patient monitor 130 and/or other data source via the network 150 for viewing and/or processing at the workstation 110.

At step 230, the retrieved data is viewed and/or processed at the workstation. Processing may include displaying, reporting, aggregating, storing, and/or otherwise processing the data, for example. For example, retrieved vital sign data is displayed for the user at the workstation 110. The data may be used in preparation for a procedure involving the patient, for example. Alternatively, data for a plurality of patients may be collected at the workstation 110 via monitors 120, 130.

One or more of the steps of the method 200 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for monitoring patient physiological data, said method comprising:
selecting a patient monitor via a mobile physiological monitoring workstation;
acquiring physiological data from said patient monitor, said data transmitted over a network from said patient monitor to said mobile physiological monitoring workstation without a dedicated connection between said mobile physiological monitoring workstation and said patient monitor; and
displaying said physiological data at said mobile physiological monitoring workstation.

2. The method of claim 1, wherein said mobile physiological monitoring workstation comprises a wireless mobile physiological monitoring workstation.

3. The method of claim 1, further comprising correlating said physiological data from a plurality of patient monitors.

4. The method of claim 1, wherein said physiological data comprises patient vital sign data.

5. The method of claim 1, wherein said physiological data comprises waveform signal data.

6. The method of claim 5, wherein said waveform signal data comprises at least electrocardiogram waveform data.

7. The method of claim 1, wherein said selecting step further comprises selecting among a plurality of patient monitors, wherein said plurality of patient monitors comprise wired and wireless patient monitors.

8. The method of claim 7, wherein a wireless access point is used for wireless communication with a wire-based network.

9. The method of claim 1, wherein said acquiring step further comprises acquiring data from said monitor before, during and after a medical procedure on a patient.

10. A patient monitoring system, said system comprising:
a plurality of patient monitors, said plurality of patient monitors communicating via at least one of wired and wireless communication links, each of said plurality of patient monitors capable of obtaining physiological data for a patient;
a wireless mobile physiological monitoring workstation receiving physiological data; and
a network enabling exchange of physiological data from said plurality of patient monitors and said workstation,
wherein said workstation may select one or more of said plurality of patient monitors from which to receive physiological data for the patient.

11. A patient monitoring system, said system comprising:
at least one patient monitor capable of obtaining and transmitting physiological data for a patient;
a mobile physiological monitoring workstation receiving physiological data; and
a network enabling exchange of physiological data between said at least one patient monitor and said workstation,
wherein said workstation may select one or more of said at least one patient monitor from which to receive physiological data for the patient.

12. The system of claim 11, wherein said mobile physiological monitoring workstation comprises a wireless mobile physiological monitoring workstation.

13. The system of claim 11, further comprising a plurality of patient monitors, said plurality of patient monitors communicating via at least one of wired and wireless communication links, each of said plurality of patient monitors capable of obtaining physiological data for a patient.

14. The system of claim 11, further comprising correlating said physiological data from a plurality of patient monitors.

15. The system of claim 11, wherein said physiological data comprises patient vital sign data.

16. The system of claim 11, wherein said physiological data comprises waveform signal data.

17. The system of claim 16, wherein said waveform signal data comprises at least electrocardiogram waveform data.

18. The system of claim 17, wherein a wireless access point is used for wireless communication with a wire-based network.

19. The system of claim 11, said physiological data is collected from said at least one patient monitor before, during and after a medical procedure on a patient.

20. The system of claim 11, wherein said workstation automatically selects one or more of said at least one patient monitor from which to receive physiological data for the patient.
